(19) United States
(12) Patent Application Publication
(10) Pub. No.: US 2014/0093572 A1
(43) Pub. Date: Apr. 3, 2014

(54) ACTIVE MATERIALS FOR PREVENTION AND TREATMENT OF FOULED SURFACES

(71) Applicants: Mitchel J. Doktycz, Knoxville, TN (US); David P. Allison, Lenoir City, TN (US); Charles F. Barnett, Knoxville, TN (US); Scott T. Retterer, Knoxville, TN (US)

(72) Inventors: Mitchel J. Doktycz, Knoxville, TN (US); David P. Allison, Lenoir City, TN (US); Charles F. Barnett, Knoxville, TN (US); Scott T. Retterer, Knoxville, TN (US)

(73) Assignee: UT-BATTELLE, LLC, Oak Ridge, TN (US)

(21) Appl. No.: 14/114,797

(22) PCT Filed: May 4, 2012
(86) PCT No.: PCT/US12/36468

§ 371 Date: Oct. 30, 2013

Related U.S. Application Data
(60) Provisional application No. 61/483,133, filed on May 6, 2011.

(51) Int. Cl. A61K 41/00

(52) U.S. Cl. CPC A61K 41/0057; A61K 41/0052
USPC 424/489; 424/649; 424/618; 424/630; 424/646; 424/400; 424/617; 424/639

(57) ABSTRACT

A method, composition and structure to treat fouling. In one embodiment, the method of treating fouling includes providing a structure including a first component of a base material and a second component of an energetically activated nanostructure, and applying a stimuli to the structure that effectuates an increase or decrease in the temperature of the energetically activated nanostructure. The increase or decrease in the temperature of the energetically activated nanostructure modifies the chemical and/or mechanical properties of the base material. The modifications to the chemical and/or mechanical properties of the base material obstruct fouling of the structure.

### CROSS-REFERENCE TO RELATED APPLICATIONS

This application claims the benefit of priority from U.S. Provisional Application No. 61/483,133, filed May 6, 2011, the content of which, in its entirety, is incorporated by reference.

### STATEMENT REGARDING FEDERALLY SPONSORED RESEARCH OR DEVELOPMENT

This invention was made with government support under Contract Number DE-AC05-00OR22725 between the United States Department of Energy and UT-Battelle, LLC. The U.S. government has certain rights in this invention.

### FIELD OF THE INVENTION

The present disclosure relates to methods for the prevention and treatment of fouled surfaces, as well as materials for the prevention and treatment of fouled surfaces.

### BACKGROUND

Modern medicine is served by a variety of man-made devices that are inserted into individuals for a variety of reasons including medical tests, drug treatments, cosmetic applications and long term corrective measures. Perhaps the most common implanted devices are vascular and urinary catheters, such as vascular catheters used in kidney dialysis. Other applications of vascular catheters would include the introduction of medications, nourishment, and drugs into patients over an extended period of time. Urinary catheters are also used to both monitor and assure urinary output. Shunts may be inserted into patients to move liquid from one part of the body to another, examples being the ventriculoperitoneal shunt used to relieve intracranial pressure by moving excess cerebrospinal fluid from the brain into the peritoneal cavity. Cosmetic treatments that employ man made devices include breast implants that place manufactured material into the body. Artificial joints including artificial knees, shoulders, hips, and ankles are also routinely implanted by orthopedic surgeons.

Each of above man-made devices have a common problem in that they may become infected. The mainstay treatment of bacterial infections of implanted devices is antibiotic use. However, antibiotic resistant bacterial infections are common and difficult to treat. Additionally, infectious bacteria frequently form biofilms on devices and can be difficult to treat. Antibiotic-based treatment of biofilms are generally ineffective due to the inability of the antibiotic to penetrate the biofilm. Biofilm formation proceeds through stages and begins with surface conditioning through adsorption of materials, e.g., organics and biomolecules. These materials facilitate the attachment of microbial cells. After attachment to the surface, extracelluar polymeric substances (EPS) are produced that can regulate the exchange of soluble materials and structuring of the biofilm. (The presence of EPS is thought to be responsible for the reduced efficacy of antibiotic treatments.) After bacterial attachment, colonization proceeds as bacteria divide and produce EPS to form the biofilm. The above phenomena may be referred to as fouling. Technologies for preventing fouling can potentially eliminate complications associated with microbial infection.

### SUMMARY

In one aspect, the present disclosure provides a method to prevent and treat fouling. In one embodiment, the method may include providing a structure composed of a first component of a base material and a second component of an energetically activated nanostructure. To prevent fouling, a stimuli is applied to the structure that effectuates an increase or decrease in the temperature of the energetically activated nanostructure. The increase or decrease in the temperature of the energetically activated nanostructure modifies the chemical and/or mechanical properties of the base material. The modifications to the chemical and/or mechanical properties of the base material obstruct fouling of the structure.

In another aspect, a composite structure is provided that includes an energetically activated nanostructure to prevent fouling. In one embodiment, the composite includes a matrix phase of a first material composition, and a dispersed phase of an energetically activated nanostructure of a second material composition. The dispersed phase of the energetically activated nanostructure is intermixed with the matrix phase of the first material composition. The dispersed phase of the energetically activated nanostructure when activated modifies the matrix phase to obstruct fouling of the composite.

In another aspect, a coated structure is provided that includes an energetically activated nanostructure to prevent fouling. In one embodiment, the coated structure includes a geometry of a base structure, and a coating of an energetically activated nanostructure on a surface of the geometry of the base structure. When activated, the energetically activated nanostructure modifies the geometry of the base structure to obstruct fouling of the coated structure.

### BRIEF DESCRIPTION OF THE DRAWINGS

The following detailed description, given by way of example and not intended to limit the disclosure solely thereto, will best be appreciated in conjunction with the accompanying drawings, wherein like reference numerals denote like elements and parts, in which:

FIG. 1 is a perspective view of a vascular catheter, in accordance with one embodiment of the present disclosure.

FIG. 2 is a side cross-sectional view of a surface of the vascular catheter that is depicted in FIG. 1, wherein the vascular catheter is a composite including a matrix phase of a first material composition, and a dispersed phase of an energetically activated nanostructure of a second material composition, wherein when activated the energetically activated nanostructure modifies the matrix phase to obstruct fouling, in accordance with one embodiment of the present disclosure.

FIG. 3A-3C are micrographs depicting core-shell nanoparticles, in which the core of the nanoparticles is composed of silicon, and the shell is a composed of gold, in accordance with one embodiment of the present disclosure.

FIG. 4 is a micrograph depicting gold nanorods, in accordance with one embodiment of the present disclosure.

FIG. 5 is a plot of the absorption of gold nanorods as a function of light wavelength, in accordance with one embodiment of the present disclosure.

FIG. 6 is a side cross-sectional view of a laminated structure including sheets composed of an energetically activated nanostructure, in accordance with one embodiment of the present disclosure.

FIGS. 7A and 7B are a side-cross sectional views of a coated surface of the vascular catheter that is depicted in FIG. 1, wherein the coating includes an energetically activated nanostructure that modifies the surface of the vascular catheter to obstruct fouling, in accordance with one embodiment of the present disclosure.

FIGS. 8A and 8B are side cross-sectional views depicting the obstruction of microorganisms that can foul a surface by mechanical motion in response to stimuli of optical waves, in accordance with one embodiment of the present disclosure.

### DETAILED DESCRIPTION

Detailed embodiments of the present disclosure are described herein; however, it is to be understood that the disclosed embodiments are merely illustrative of the compositions, structures and methods of the disclosure that may be embodied in various forms. In addition, each of the examples given in connection with the various embodiments are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the compositions, structures and methods disclosed herein. References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. For purposes of the description hereinafter, the terms “upper”, “lower”, “right”, “left”, “vertical”, “horizontal”, “top”, “bottom”, and derivatives thereof shall relate to the disclosed structures, as they are oriented in the drawing figures.

The present disclosure in one embodiment relates to preventing fouling, such as preventing the attachment and growth of microorganisms on a surface, e.g., the surface of a medical implant device. As used herein, the term “fouled” or “fouling” denotes the deposition or growth of a material on a surface. Formation of microorganisms on a surface is a form of biofouling. The terms “Biofouled” or “Biofouling” denote the deposition and/or growth of microorganisms on a surface. In one example, biofouling of the surface of the medical implant device includes microbial interactions with the surface of the medical implant device that can progress to form stable biofilms that can lead to medical complications, such as infection in the subject receiving the medical implant. In addition to biofouling, the present disclosure also relates to organic, inorganic and particle fouling. Inorganic fouling may include the deposition of an inorganic material, such as silt, clay or humic particles, whereas organic fouling may include deposition of organic materials, such as fat, oil, proteins, or biomolecules. Particle fouling may include precipitation of inorganic crystals.

In one embodiment, to prevent fouling a structure is provided that is composed of a first component of a base material and a second component of an energetically activated nanostructure. The term “energetically activated nanostructure” denotes a structure including nanoparticles that in response to a stimulus experience an increase or decrease in temperature. A “nanoparticle” is a particle having a dimension, such as a radius or longest axis, of 1000 nm or less. In one example, the energetically activated nanostructure includes at least one dimension, e.g., at least one of the height (y-axis), width (x-axis) or length (z-axis), that is less than 100 nm.

By “stimulus” or “stimuli” it is meant an application of energy, such as optical waves or alternating electromagnetic fields, which causes an energetically activated physical or chemical change in the nanostructure. In one embodiment, the energetically activated nanostructure in response to the stimuli cause a mechanical or chemical change in the first component of the structure that is composed of the base material. The mechanical change can be mechanical motion, whereas the chemical change may be a chemical reaction or a phase change in the base material. The mechanical and/or chemical changes in the base material provide local changes in the surface properties of the structure that are disruptive to the adsorption of fouling agents that can result in one of biofouling, organic fouling, inorganic fouling and/or particle fouling of the structure. For example, the mechanical and/or chemical changes in the base material can vary with time, so that they are disruptive to fouling agents.

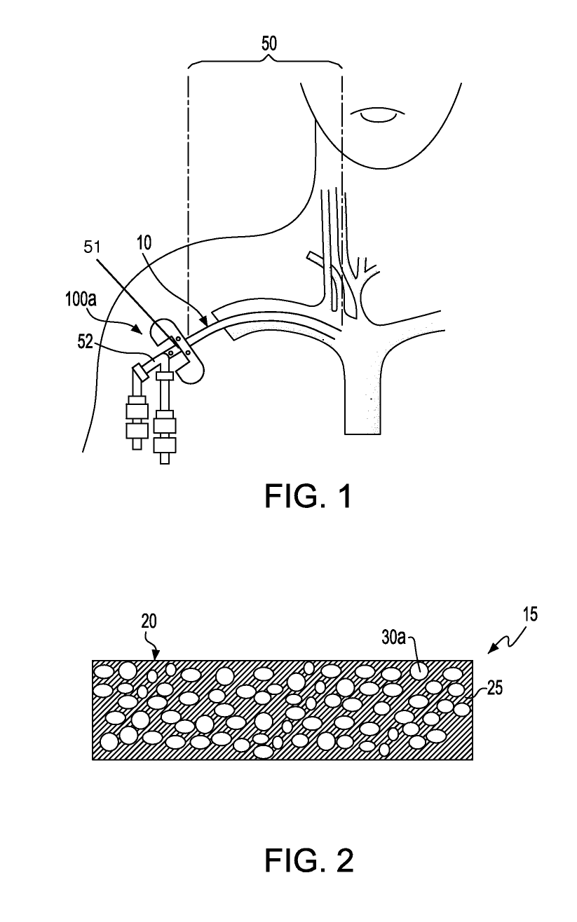

For descriptive purposes, the structure may be a vascular catheter 100A, as depicted in FIG. 1, in which the first component of the base material is a structural material that provides the geometry of at least one component of the vascular catheter 100A, and the second component of energetically activated nanostructure includes nanoparticles that in response to a stimuli of optical waves or magnetic fields experiences an increase or decrease in temperature. In one example, the increases or decrease in the temperature of energetically activated nanostructure provide an expansion or contraction of the base material, which results in a mechanical motion of the catheter surface 10 that obstructs fouling. More specifically, in one example, the mechanical motion is disruptive to the adsorption of the pathogenic microorganisms that may cause fouling on the surface 10 of the vascular catheter 100. The mechanical motion of the catheter surface 10 prevents colonization of the surface by pathogenic microorganisms, and the mechanical motion of the catheter surface 10 disrupts biofilms that may be present thereon so that they may be eliminated. Although FIG. 1 depicts a vascular catheter 100A, the methods, compositions and structures that are disclosed herein are applicable to any medical implant device including, but not limited to, other catheters, such as urinary catheters, shunts, artificial joints, dental implants, cosmetic implants and combinations thereof. Although the following description is directed to medical implant devices, it is not intended that the present disclosure be limited to only medical implant devices, as the methods, structures and compositions disclosed herein are applicable to any surface that may be fouled. For example, transportation applications have also been contemplated, such as naval, space and aerospace applications. Additionally, applications in air or water purification can be considered.

Referring to FIGS. 1 and 2, in one embodiment, the vascular catheter 100A is composed of a composite material 15. FIG. 2 is a side cross sectional view of the surface 20 of the vascular catheter 100A that is depicted in FIG. 1, wherein the surface 20 having the anti-fouling performance is composed of a composite material 15. Referring to FIG. 2, the composite material 15 is a combination of two or more materials, i.e., constituents, that retain their identities, as they do not dissolve or merge completely into one another, but act in concert. Normally, the components exhibit an interface between one another. In one embodiment, the geometry of the vascular catheter 100A is provided by a first component of a base material 25 that may be referred to as a matrix phase. The base material 25 provides the structure material of the vascular catheter 100A. The surface 20 of the vascular catheter 100A that is depicted in FIG. 2 also includes a dispersed phase of the energetically activated nanostructure 30a of a second material composition that is intermixed with the matrix of the first material composition, i.e., base material 25. By “dispersed” it is meant that the nanoparticles that provide the energetically activated nanostructure 30a are at least present throughout the surface of the structure at which anti-fouling performance is desired. The dispersed phase of the energetically activated nanostructure 30a when activated modifies the base material 25, i.e., matrix phase, to obstruct fouling of the surface 20 of the vascular catheter of the composite material 15.

For example, referring to FIGS. 1 and 2, in one embodiment in which the structure is a vascular catheter 100A, the nanoparticles that provide the energetically activated nanostructure 30a may be positioned in the sub-cutaneous portion 50 of the vascular catheter 100 and may extend externally at the point of insertion 51. Although, in some embodiments the nanoparticles that provide the energetically activated nanostructure 30a are dispersed in only the surfaces that anti-fouling performance is desired, the nanoparticles of the energetically activated nanostructure 30a may be dispersed throughout the entirety of the base material 25 (also referred to as matrix phase) that provides the structural geometry of the structure provided by the composite material 15, such as the composite vascular catheter 100A.

A number of nanomaterials may be considered for providing the energetically activated nanostructure 30a. For example, the nanoparticles that provide the energetically activated nanostructure 30a may be metal or metal oxide particles that are composed of gold, silver, copper, iron, palladium, platinum, and a combination thereof. The metal nanoparticles may be composed of a single composition, or may include a core composition and a coating composition. Metal nanoparticles that are spherical in shape and composed of single composition may be referred to as a metal nanosphere. Nanoparticles having a metal coating on a semiconductor, dielectric, or metallic core may be referred to as core-shell nanoparticles. In one embodiment, when the metal nanoparticles are core-shell nanoparticles, the core may be composed of a semiconductor, metal, metal oxide or dielectric material. For example, the semiconductor material that provides the core composition may be silicon (Si) or silica (SiO₂). The coating composition may be composed of a metal, such as gold. Nanoparticles having a hollow interior are referred to as nanoshells.

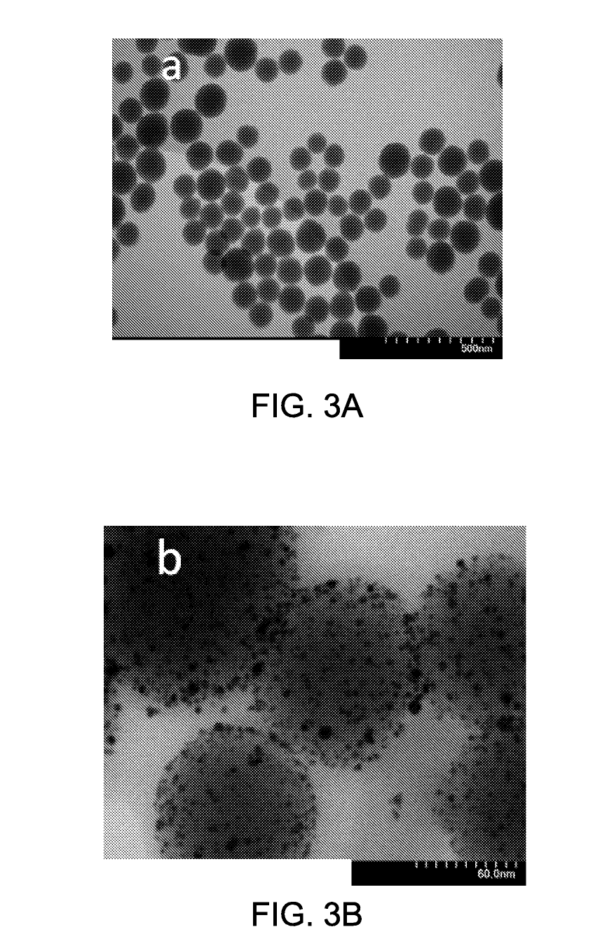

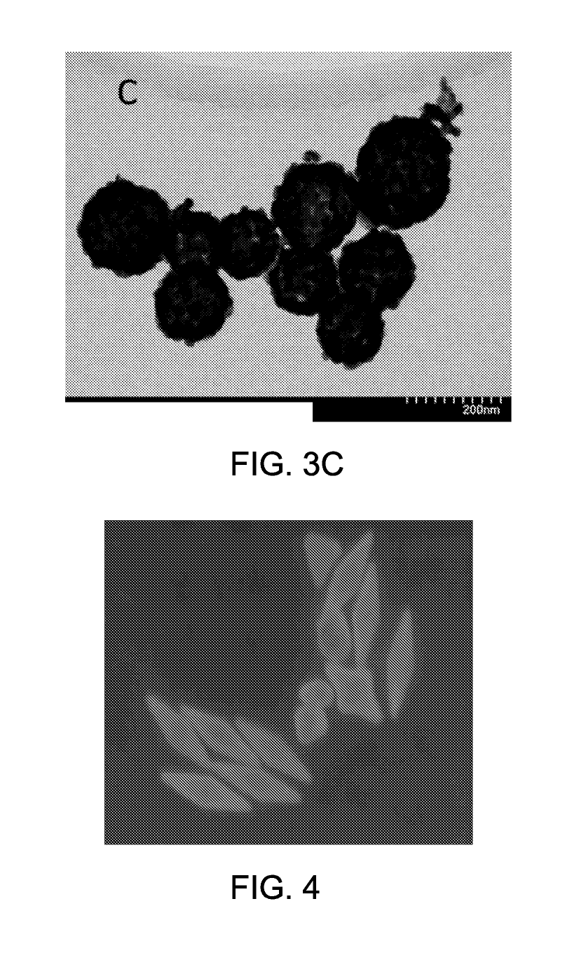

FIG. 3A-3C are micrographs depicting core-shell nanoparticles, in which the core of the nanoparticles is composed of silica, and the shell is a composed of gold. FIG. 3A depicts the formation of silica nanoparticles from the addition of tetraethylorthosilicate (TEOS) to ammonium hydroxide (NH₄OH) in ethanol. FIG. 3B depicts adding aminopropyltrimethoxy silane to mixture of the silica nanoparticles, wherein amino functionality is introduced to the silica nanoparticles allowing colloidal gold to nucleate the surface of the silica nanoparticles. Referring to FIG. 3C, the further addition of a chloroauric acid growth solution allows a shell of gold to form on the silica nanoparticles to provide a core-shell nanoparticles.

The geometry of the nanoparticles that provide the energetically activated nanostructure 30a may be substantially spherical, platelet, rod-shaped, or a combination thereof. In one embodiment, the longest axis of the nanoparticles that provide the energetically activated nanostructure 30a may range from 1 nm to 5000 nm. In another embodiment, the longest axis of the nanoparticles that provide the energetically activated nanostructure 30a may range from 100 nm to 2500 nm. FIG. 4 depicts one embodiment of gold nano-rods, i.e., rod-shaped nanoparticles that are suitable for providing the energetically activated nanostructure 30a. Controlled synthesis of gold nano-rods, as depicted in FIG. 4, may be accomplished by incorporating gold seed solution into a rod-shaped micellar environment. The growth phase for the synthesis of the gold nano-rods is controlled by limiting the concentration of choloroauric acid.

In one embodiment, the energetically activated nanostructure 30a includes nanoparticles that when subjected to a stimuli, such as an alternating electromagnetic field or optical wave (light), display plasmon resonances. Plasmon resonance occurs as a result of collective oscillations of conduction electrons of the energetically activated nanostructure 30a. The factors that collectively lead to the oscillations of the conductive electrons include acceleration of the conduction electrons by the electric field of incident radiation (alternating electromagnetic field or optical wave), presence of restoring forces that result from the induced polarization in both the particle and the surrounding medium, and confinement of the electrons to dimensions smaller than the wavelength of light. The electric field (alternating electromagnetic field or optical wave) displaces the particle's electrons from equilibrium and, in turn produces a restoring force that results in oscillatory motion of the electrons with a characteristic frequency. The plasmon resonance of metal nanoparticles is highly tunable and depends on the size and shape of the nanoparticle. With the absorption of light or the application of the alternating electromagnetic field, the nanoparticles of the energetically activated nanostructure 30a may release heat or undergo chemical or physical changes.

In some examples, in which the nanoparticles that provide the energetically activated nanostructure 30a exhibit the above described plasmon resonance in response to a stimuli of optical waves, the nanoparticles may be composed of gold, silver, platinum, palladium, copper or a combination thereof. The absorption properties of these nanoparticles can be tuned to absorb light at wavelengths in the visible light region, the near infrared region (NIR), and the infrared region (IR). The term “visible light region” denotes light wavelengths of less than 780 nm. NIR light wavelengths range from 780 nm-3000 nm. The IR light wavelengths range from greater than 3000 nm to 1000 μm, which include mid infrared and far infrared light wavelengths.

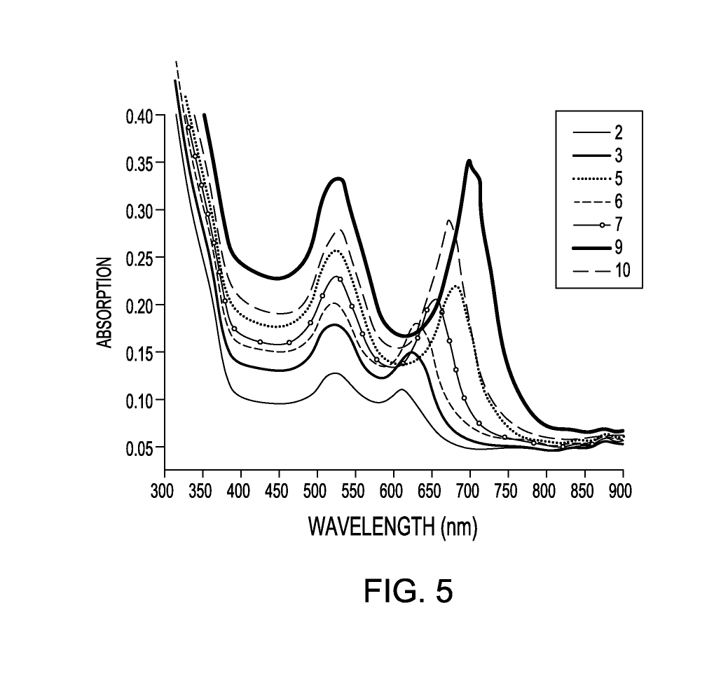

One factor that may be utilized to tune the nanoparticles to absorb a specified range of optical (light) wavelengths is the size of the nanoparticles. In core-shell nanoparticles by varying core size and shell thickness the particles can be tuned to absorb light from the optical to the infrared region. For example, with nanoparticles having a core composed of a semiconductor or dielectric material, such as silicon oxide, and a coating of a metal, such as gold, when the cores are relatively small the peak plasmon resonance is in the visible or NIR wavelength regions. However, increasing the size of the core and reducing the thickness of the coating may shift the peak plasmon resonance into the IR wavelength region. Another factor that may be utilized to tune the nanoparticles to absorb a specified range of light wavelengths is the geometry of the nanoparticles. For example, the absorbed optical wavelengths of a gold nanosphere may be within the visible range, and the absorbed optical wavelengths of a gold nanoshell may be within the NIR wavelength range. In comparison to the gold nanospheres and gold nanoshells, gold nanorods may shift the absorbed optical wavelengths into the IR range. With gold nanorods the aspect ratio of length to width causes a shift in absorbance. FIG. 5 is a plot depicting the absorption of light wavelengths by gold nanorods that are similar to those that are depicted in FIG. 4. Nanorods with lower aspect ratios absorb light at a lower wavelength than do nanorods where the length exceeds the width significantly, as shown in FIG. 5.

With the absorption of light, i.e., optical wavelengths, the nanoparticles that provide the energetically activated nanostructure 30a release heat. In one embodiment, in response to a stimuli of optical waves, the temperature of the nanoparticles may increase or decrease by +/−1000° C. from an ambient temperature that ranges from 20° C. to 40° C. In another embodiment, in response to a stimuli of optical waves, the temperature of the nanoparticles may increase or decrease by +/−100° C. from an ambient temperature that ranges from 35° C. to 40° C.

In one embodiment, the nanoparticles that provide the above described photothermal properties include silica (SiO₂) particles having a gold coating. Silica particles may be formed from tetraethyl orthosilicate (TEOS) (Si(OC₂H₅)₄) reduced in ammonium hydroxide (NH₄OH) in ethanol (C₂H₅OH), as depcited in FIG. 3A. The surface of the silica partices may be terminated with amino groups by reaction with aminopropyltriethoxysilane (H₂N(CH₂)₃Si(OC₂H₅)₃) in ethanol. Gold colloids having a diameter or less than 5 nm may be formed using a synthesis method based on the reduction of chloroauric acid (HAuCl₄) in water containing NaOH and the reducing agent tetrakis(hydroxymethyl)phosphonium chloride (THPC). The aminated silica particles may be added to the gold colloid suspension, in which the gold colloid adsorbs to the amine groups on the silica surface resulting in a silica nanoparticles covered with gold colloid, as shown in FIG. 3B. Gold-silica nanoshells may then be grown by reacting chloroauric acid (HAuCl₄) with the silica-colloid nanoparticles in the presence of formaldehyde, as shown in FIG. 3C. In one embodiment, this process reduces additional gold onto the adsorbed colloid, which act as nucleation sights, causing the surface colloid to grow and coalesce with the neighboring colloid, forming a complete metal shell. The nanoparticles formed by the above-described method had a core with a radius ranging from 45 nm to 65 nm, and a shell having a thickness ranging from 5 nm to 15 nm. In one example, a nanoparticle is provided that includes a silica core having a radius of about 55 nm, and a coating composed of gold having a thickness of about 10 nm. The nanoparticles were then suspended in deionized water (DI). In one example, the nanoparticles may be present in the suspension in a concentration ranging from 1.5×10⁹ nanoparticles per ml to 1.5×10¹¹ nanoparticles per ml.

In another embodiment, the nanoparticles of the energetically activated nanostructure 30a may be silver (Ag) nanospheres. The silver nanospheres may be provided by the reduction of a supersaturated aqueous solution of silver oxide (Ag₂O) by hydrogen gas. In one embodiment, the aqueous solution is maintained at a temperature ranging from 60° C. to 80° C., e.g., 70° C., and the hydrogen gas is pressurized at 5 psi to 15 psi above atmosphere, e.g., 10 psi above atmosphere. In this embodiment, the particle size may range from 10 nm to 200 nm, depending on the reaction time, with a standard deviation of particle size between 5% and 8%. The silver nanoparticles may be present in the solution in a concentration ranging from 1×10¹⁰ cm⁻³ to 1×10¹³ cm⁻³.

In addition to nanoparticles having a composition that is activated by optical stimuli, the composition of the nanoparticles of the energetically activated nanostructure 30a may be selected to generate heat in response to a magnetic stimuli. Examples of materials that may be activated to generate heat using a magnetic stimuli, such as an alternating electromagnetic field, include magnetite nanoparticles (Fe₃O₄). Other examples of materials that generate heat when subjected to an electromagnetic field include composite particles of cobalt (Co), lanthium (La), strontium (Sr) and manganese (Mn). In some embodiments, these materials have superparamagnetic properties, when the individual particle size is less than 15 nm and composed of a single magnetic domain. Superparamagnetism is a form of magnetism that appears in nanoparticles having a single magnetic domain, in which the magnetizm can randomly change direction under the influence of an alternating magnetic field. Nanoparticles having a composition that is activated by magnetic stimuli can be inductively heated by a magnetic field generated by an alternating current. Heating can be attributed to friction of the particle rotating in the magnetic field or to Néel relaxation where energy applied to the particle, by the alternating magnetic field, allows the magnetic moment in the particle to overcome the energy barrier. This energy is then dissipated as heat when the particle moment relaxes to its equilibrium orientation. It is noted that larger particles of multiple superparamagnetic particles can also be synthesised.

In one embodiment, in response to a stimuli of a magnetic field the temperature of the nanoparticles may increase or decrease by +/−1000° C. from an ambient temperature that ranges from 20° C. to 40° C. In another embodiment, in response to a stimuli of a magnetic field the temperature of the temperature of the nanoparticles may increase or decrease by +/−100° C. from an ambient temperature that ranges from 35° C. to 40° C.

In the embodiments that are depicted in FIGS. 1 and 2, the nanoparticles that provide the energetically activated nanostructure 30a of the second component, i.e., dispersed phase, of the composite structure are integrated or intermixed with the first component of a base material 25, i.e., matrix phase. The base material 25 may be composed of a polymeric composition, but other materials have been contemplated including, but not limited to ceramics, glass, and metals. In the embodiment depicted in FIGS. 1 and 2, in which the composite structure is a catheter 100A, the base material 25 is a polymeric composition having biocompatibility and physical properties (tensile strength and elasticity) that are suitable for use in the subject in which the catheter 100A is being employed. For example, the polymeric composition may be selected to withstand pressures on the order of 110,000 to 300,000 psi at the proximal end of the catheter 100A, and pressures ranging from 2000 to 10,000 psi at the distal end of the catheter 100A.

In one embodiment, the polymer composition employed for the base material 25 of the catheter 100A is a polyurethane. Examples of polyurethanes that are suitable for the base material 25 include polycarbonate-based polyurethanes, polyether-based polyurethanes and polyester-based polyurethanes. In another embodiment, the polymer composition employed for the base material 25 of the catheter 100A is a polyamide and polyamide block copolymer. In one example, in which the base material 25 is composed of a polyamide, the polyamide composition may be one of nylon 11 and nylon 12 and its other block copolymers. In a further embodiment, the polymer composition employed for the base material 25 of the catheter 100A is a fluoropolymer. One example, of a fluoropolymer that is suitable for the base material 25 of the catheter is polytetrafluoroethylene (PTFE). In yet another embodiment, the polymer composition employed for the base material 25 of the catheter 100A is a polyolefin, such as high-density polyethylene. Other examples of polymeric compositions that are suitable for the base material 25 of the catheter 100A include polyurethane, silicone, latex, polyvinyl chloride (PVC), polyimides and polyetheretherketone (PEEK).

Although, the base material 25 has been described as a polymeric material, the present disclosure is not so limited, any structural material introduced into the body may be employed, so long as the structure material experiences a physical or chemical response to the energetically activated nanostructure 30a which obstructs fouling of the surface of the structure that is composed of the base material 25. Examples of ceramic materials that are suitable for the base material 25 include aluminum oxide and tin oxide. Examples of glass materials that are suitable for the base material 25 include borosilicate glass.

The nanoparticles that provide the energetically activated nanostructure 30a and the base material 25 can be cointegrated using a number of techniques. Depending upon the manufacturing approach, multiple types and/or sizes of nanoparticles can be combined in a single structure. Further, the particle density could be controlled in order to effectively tune the dynamic chemical and physical properties of the doped material. For example, a solution of nanoparticles that provides the energetically activated nanostructure 30a can be dispersed within a polymer melt and either molded, casted and/or extruded to create a device, such as the catheter 100A depicted in FIG. 1. The solution of nanoparticles may be suspended in an aqueous solution or in a solvent, in which the concentration of nanoparticles ranges from 1×10⁹ particles per ml to 1×10¹⁵ particles per ml. In one embodiment, the solution of nanoparticles is selected to provide an average particle spacing the nanoparticles ranging from 50 nm to 1 micron.

Plastics extrusion is a process in which raw plastic material is melted and formed into a continuous profile through a two-dimensional die. Casting is a manufacturing process by which a liquid material is usually poured into a mold, which contains a hollow cavity of the desired shape, and then allowed to solidify. One method of plastic molding suitable for forming the mixture of the polymer melt and the nanoparticles that provide the energetically activated nanostructure 30a includes injection molding. Injection molding is a process for producing parts from both thermoplastic and thermosetting plastic materials. Material is fed into a heated barrel, mixed, and forced into a three dimensional mold cavity where it cools and hardens to the configuration of the mold cavity.

The mold, casting or extrusion die of the above described methods may have a geometry that provides at least one component of the catheter 100A that is depicted in FIG. 1. In other embodiments, the mold, casting or extrusion die is selected to form at least one component of a shunt, artificial joint, dental implant, cosmetic implant or other medical implant device. In one embodiment, the energetically activated nanostructure 30a is dispersed throughout the entirety of the base material 25 in a concentration ranging from 1×10⁹ nanoparticles/cm³ to 1×10¹⁵ nanoparticles/cm³. In one embodiment, the average particle spacing between the nanoparticles of the energetically activated nanostructure 30a is less than 1 micron. In another embodiment, the energetically activated nanostructure 30a is dispersed throughout the entirety of the base material 25 in a concentration ranging from 1×10¹² nanoparticles/cm³ to 1×10¹⁵ nanoparticles/cm³. In one embodiment, the average particle spacing between the nanoparticles of the energetically activated nanostructure 30a ranges from 50 nm to 1 micron.

Although, it is typical for the energetically activated nanostructure 30a to be dispersed throughout the entirety of the base material 25 in structures that are extruded, casted or molded, in some embodiments the energetically activated nanostructure 30a may be dispersed only on the surfaces of the structure being formed that will be subjected to conditions that are conducive to fouling. When employing a mold, casting or extrusion method, a surface containing the dispersed phase of the energetically activated nanostructure 30a may be co-molded onto a core that is composed of only the base material 25. In this embodiment, the surface containing the dispersed phase of the energetically activated nanostructure 30a may be molded onto the core of the structural material from a polymeric melt that has been mixed with a solution of nanoparticles. In this example, the nanoparticles of the energetically activated nanostructure 30a are present in the anti-fouling surface in a concentration ranging from 1×10⁹ nanoparticles/cm³ to 1×10¹⁵ nanoparticles/cm³, wherein the nanoparticles of the energetically activated nanostructure 30a are not present in the core of the structural material. In one embodiment, the average particle spacing between the nanoparticles of the energetically activated nanostructure 30a ranges from 50 nm to 1 micron.

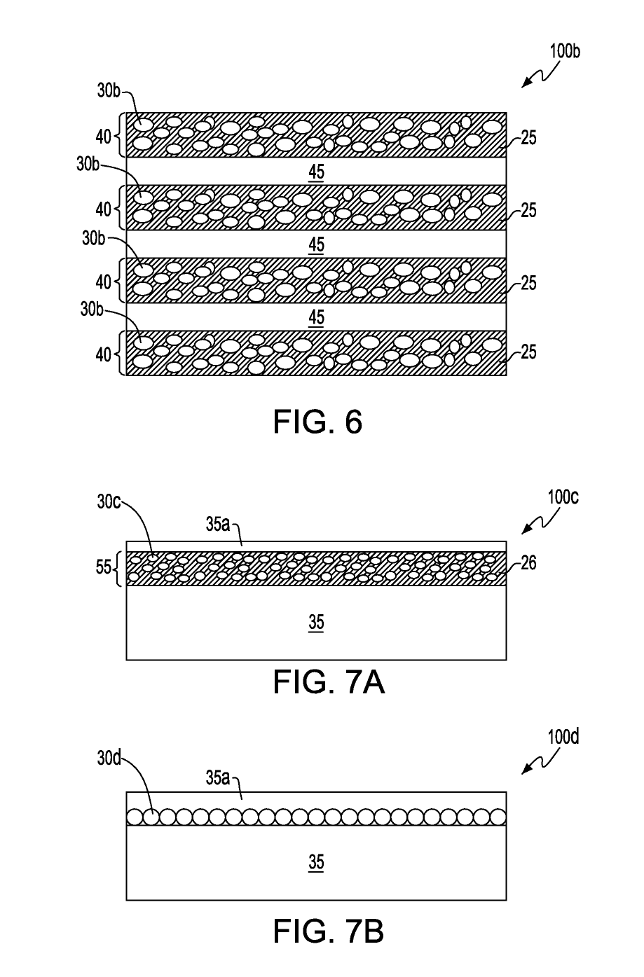

Referring to FIG. 6, in yet another embodiment, sheets 40 of base material 25 including nanoparticles that provide the energetically activated nanostructure 30b are combined with other materials, such as an adhesive 45, to create a laminated structure 100B. The sheets may be extruded from a polymer melt including nanoparticles intermixed therein to provide the energetically activated nanostructure 30b. In another embodiment, the sheets 40 may be formed by spin casting the polymer melt including nanoparticles for the energetically activated nanostructure 30b. In yet another embodiment, the sheets may be implanted with the nanoparticles for the energetically activated nanostructure 30b following formation. The nanoparticles that provide the energetically activated nanostructure 30b depicted in FIG. 6 are similar to the nanoparticles that provide the energetically activated nanostructure 30a depicted in FIG. 2. Therefore, the above description of the nanoparticles of the energetically activated nanostructure 30a that are described above with reference to FIG. 2 is applicable to the nanoparticles of the energetically activated nanostructure 30b that are depicted FIG. 6. Further, the above description of the base material 25 that is depicted in FIG. 2 is applicable for the base material 25 of the sheet 40 that is depicted in FIG. 6. Each sheet 40 may have a thickness ranging from 30 nm to 2 mm. The adhesive 45 used to join the sheets 40 of the base material 25 may include an epoxy-based resin, parylene, polyimide, as well as other photodefinable or etchable polymer materials, and combinations thereof. Other adhesives 45 may include silicone, polyimide and combinations thereof. It is noted that any number of sheets 40 and layers of adhesive 45 may be employed to provide the laminated structure 100B.

In some embodiments, the laminated structure 100B depicted in FIG. 6 may be shaped to provide a medical implant structure, such as a component of the catheter 100A that is illustrated in FIG. 1. Referring to FIG. 6, the laminated structure 100B may be shaped using micro-fabrication techniques, such as photolithography and etch processes. For example, a layer of photoresist (not shown) may be deposited on the upper surface of the laminated structure 100B to provide an etch mask using a deposition process, such as spin on deposition, chemical vapor deposition (CVD) or a combination therefore. A photoresist etch mask can be produced by applying a photoresist layer to a surface of the laminated structure 100B, exposing the photoresist layer to a pattern of radiation, and then developing the pattern into the photoresist layer utilizing a resist developer. Following development, the remaining portions of the photoresist layer provide the etch mask, wherein the portions of the laminated structure 100B that are exposed by developing the exposed photoresist layer are subsequently removed by an etch process that is selective to the etch mask. As used herein, the term “selective” in reference to a material removal process denotes that the rate of material removal for a first material is greater than the rate of removal for at least another material of the structure to which the material removal process is being applied. In some examples, the selectivity may be greater than 100:1. In some embodiments, the etch may remove the exposed portion of the sheet 40 selective to the adhesive 45, and vice versa. The selective etch may be an anisotropic etch or an isotropic etch. An anisotropic etch process is a material removal process in which the etch rate in the direction normal to the surface to be etched is greater than in the direction parallel to the surface to be etched. In comparison to an anisotropic etch, an isotropic etch is non-directional. The anisotropic etch may include reactive-ion etching (RIE). Other examples of anisotropic etching that can be used at this point of the present disclosure include ion beam etching, plasma etching or laser ablation. An isotropic etch may be provided by a wet chemical etch. Following etching, the etch mask may be removed.

It is noted that any number of etch and photolithography process sequences may be conducted to provide a three dimensional shape from the laminate 100B depicted in FIG. 6. For example, the laminate 100B that is depicted in FIG. 6 may be shaped into architectures, such as mesh nets, or coils. These more complex geometries may be applied to coat prosthetic joints, shunts or other three-dimensional implanted devices, and can be used to tune dynamic thermal properties or optical properties.

FIGS. 7A and 7B depict one embodiment of a coated structure 100C, 100D including a geometry of a base structure 35, and a coating 55 of an energetically activated nanostructure 30c, 30d on a surface of the geometry of the base structure 35. The coating 55 of energetically activated nanostructures 30c, 30d may be recoated with a thin overcoat layer of material corresponding to the base material, i.e., material of the base structure 35. When activated, the energetically activated nanostructure 30c, 30d modifies the geometry of the overcoat material that is present atop the energetically activated nanostructure 30c, 30d to obstruct fouling of the coated structure. The nanoparticles that provide the energetically activated nanostructure 30c, 30d may be adsorbed onto the surface of the geometry of the base structure 35 or chemically attached to the surface of the geometry of the base structure 35. The geometry of the base structure 35 may be any structure in which anti-fouling performance is desired, such as the catheter 100A that is depicted in FIG. 1. In the embodiments in which the base structure 35 is a catheter 100A, the base structure 35 may be composed of a polyurethane, polyamide, polyamide block copolymer, fluoropolymer, polyolefin, polyvinyl chloride (PVC), polyimides, polyetheretherketone (PEEK), and combinations thereof. Other medical implant devices that may be coated in accordance with the present disclosure include shunts, artificial joints, dental implants, cosmetic implants or other medical implant devices.

Referring to FIG. 7A, the materials that provide the energetically activated nanostructure 30c may be deposited by chemical and physical deposition methods. Examples of chemical deposition methods include chemical vapor deposition, such as metal organic chemical vapor deposition. Examples of physical deposition methods include electron beam physical vapor deposition (EBCVD), ion plating, ion beam assisted deposition (IBAD), magnetron sputtering, pulsed laser deposition, sputter deposition, vacuum deposition and vacuum evaporation. The coating may also be deposited with chemical and electrochemical methods including anodizing, plasma electrolytic oxidation, sol gel processing and plating, i.e., electroless plating or electroplating. The coating 55 of the energetically activated nanostructure 30c may also be sprayed onto the geometry of the base structure 35 using, e.g., thermal spray or plasma spray techniques. In yet another embodiment, the energetically activated nanostructure 30c may be coated onto the geometry of the base structure 35 using spin coating, dip coating, curtain coating, and equivalents thereof. Microfabrication techniques including photolithography and electron beam lithography may be used prior to material deposition to define a template material on top of the base structure 35 onto which the energetically activated nanostructure 30c, or coating 55 containing the energetically activated nanostructure 30c, is to be deposited. Lift-off techniques are also suitable to define a template material on top of the base structure 35. Lift-off techniques may include methods in which a lithographically defined template is removed, leaving the nanomaterials, e.g., coating 55 of energetically activated nanostructure 30c, behind in a well-defined 2 dimensional arrangement upon the base structure 35. The aforementioned processes can be repeated to create patterns consisting of multiple nanostructures of varied materials. In another embodiment, lithographic patterning of an etch mask material may be carried out following material and or nanostructure deposition, e.g., formation of a coating 55 of energetically activated nanostructures 30c. Subsequent wet chemical etching or reactive ion etching (RIE) may be used to selectively attack the exposed materials or nanostructures, leaving a well-defined two-dimensional matrix behind upon removal of the etch mask. Material deposition techniques may be used to control the height of lithographically defined architectures, and sequential deposition of multiple materials may be used to create stacks of materials with complimentary or unique properties. The controlled two-dimensional arrangement of nanostructured materials, e.g., energetically activated nanostructures, as well as the control of their individual three-dimensional architecture, within or upon the base structure will allow tuning of resonance during excitation. As previously described, subsequent microfabrication techniques may be used to define the base layer material, and assemble the defined two-dimensional structures and base layer material into more complex three-dimensional laminate architectures.

Referring to FIG. 7A, in one embodiment, a solution of nanoparticles that provides the energetically activated nanostructure 30c can be dispersed within a polymer melt and then coated onto a surface of the geometry of the base structure 35, such as the catheter 100A as depicted in FIG. 1, using a coating method, such as spin coating, dip coating or curtain coating. The polymer melt that is deposited to provide the coating 55 may be similar to the polymer melt used in the molding, casting, extrusion method that forms the composite structure 15 depicted in FIG. 2. In the embodiment depicted in FIG. 7A, the polymer component of the melt being deposited provides the matrix 26 of the coating 55 that contains the nanoparticles that provide the energetically activated nanostructure 30c. In some embodiments, the matrix 26 is composed of polyurethane, polyamide, polyamide block copolymer, fluoropolymer, polyolefin, polyvinyl chloride (PVC), polyimides, polyetheretherketone (PEEK), silicone, and combinations thereof. The nanoparticles that provide the energetically activated nanostructure 30c depicted in FIG. 7A are similar to the nanoparticles that provide the energetically activated nanostructure 30a depicted in FIG. 2. Therefore, the above description of the nanoparticles of the energetically activated nanostructure 30a that are described above with reference to FIG. 2 is applicable to the nanoparticles of the energetically activated nanostructure 30c that are depicted FIG. 7A. The coating 55 may have a thickness ranging from 10 nm to 100 μm. In another embodiment, the coating 55 may have a thickness ranging from 100 nm to 5 μm. The concentration of nanoparticles in the coating 55 that provide the energetically activated nanostructure 30c may range from 1×10⁹ particles/cm³ to 1×10¹⁵ particles/cm³. In one embodiment, the average particle spacing between the nanoparticles of the energetically activated nanostructure 30c ranges from 50 nm to 1 micron. An overcoat layer 35a is then formed atop the coating 55 of the energetically activated nanostructure 30c. The overcoat layer 35a may be composed of a polymer that is selected from consisting of polyurethane, polyamide, polyamide block copolymer, fluoropolymer, polyolefin, polyvinyl chloride (PVC), polyimides, polyetheretherketone (PEEK), silicone, and combinations thereof. The overcoat layer 35a may have the same composition or may have a different composition as the base structure 35. In one embodiment, the overcoat layer 35a may be formed using spin coating, dip coating or curtain coating.

FIG. 7B depicts yet another embodiment of a coated structure 100D, in which the energetically activated nanostructure 30d is a monolayer of functionalized nanoparticles bonded to a reactive surface of the base structure 35, such as the catheter 100A that is depicted in FIG. 1. For example, a thin film with chemical functionality could be applied to the device. In a following step, nanoparticles are introduced to the thin film, wherein the nanoparticles have been chemically functionalized to react with the functionality of the thin film. The reaction of the nanoparticles and the thin film leads to the formation of a monolayer of particles on the base structure 35, which provides the energetically activated nanostructure 30d. In one example, the thin film is provided by a chemical compound containing a thiol (S—H) group that can be placed on the surface of the base structure 35 for reacting gold or silver nanoparticles in forming the energetically activated nanostructure 30d. An overcoat layer 35a is then formed atop the energetically activated nanostructure 30d. The overcoat layer 35a may be composed of a polymer that is selected from consisting of polyurethane, polyamide, polyamide block copolymer, fluoropolymer, polyolefin, polyvinyl chloride (PVC), polyimides, polyetheretherketone (PEEK), silicone and combinations thereof. The overcoat layer 35a may have the same composition or may have a different composition as the base structure 35. In one embodiment, the overcoat layer 35a may be formed using spin coating, dip coating or curtain coating.

The various surface coating techniques that have been described above with reference to FIGS. 7A and 7B can be homogeneously or heterogeneously coated onto the surface of the base structure 35. Further, the surface coating techniques described above with reference to FIGS. 7A and 7B can be selectively applied through printing, patterning or masking techniques. These approaches can create defined nanoparticle arrangements that can control chemical or physical activities on the surface so as to prevent fouling.

In another aspect of the present disclosure a method to treat fouling is provided. The method may begin with providing a structure composed of a first component of a base material 25, 26, 35, and a second component of an energetically activated nanostructure 30a, 30b, 30c, 30d. The structure being treated may be any of the above-described structures including those depicted in FIGS. 1-7B. To obstruct fouling of the structure, a stimuli is applied that effectuates an increase or decrease in the temperature of the nanoparticles of the energetically activated nanostructure 30a, 30b, 30c, 30d. In one embodiment, the stimuli that activates the nanoparticles of the energetically activated nanostructure 30a, 30b, 30c, 30d to increase or decrease their temperature may be optical (light) waves. Activation of optically responsive nanoparticles, such as the gold nanospheres and gold nanoshells, can be accomplished using light sources operating at wavelengths optimal for nanoparticle absorption. Specifically, light of the appropriate energy can induce oscillations of the conductive electrons on the nanoparticle surface of the energetically activated nanostructure 30a, 30b, 30c, 30d, which may be referred to as surface plasmon resonance, which leads to the production of heat.

The wavelength of light absorbed by the nanoparticles that provide the energetically activated nanostructure 30a, 30b, 30c, 30d in the production of heat may range from 300 nm to 3000 nm. In another example, the wavelength of light absorbed by the nanoparticles that provide the energetically activated nanostructure 30a, 30b, 30c, 30d in the production of heat may range from 300 nm to 900 nm. In yet another example, the wavelength of light absorbed by the nanoparticles that results in the production of heat may range from 400 nm to 700 nm.

This light source can be directly coupled to the anti-fouling surface through an optical fiber to activate the energetically activated nanostructure 30a, 30b, 30c, 30d. Optically filtered, broad-spectrum lamps, lasers and diodes are all suitable optical wave sources for activating the energetically activated nanostructure 30a, 30b, 30c, 30d. NIR excitation can be focused, applied from outside the body, and sufficiently penetrable to reach surfaces including the energetically activated nanostructure 30a, 30b, 30c, 30d that are implanted within patients. In the NIR frequency range, penetration of tissue is optimal due to low optical absorption of tissue at these wavelengths making this safe for medical applications. For devices such as the catheter 100A depicted in FIG. 1, with device access outside the body, light energy can also be applied directly. For example, the lumen 52 of the catheter 100A could be used as a path for wave conduction or, alternatively, light could be conducted by the outer surface of the catheter. Light conductive material could also be incorporated with the device to facilitate operation. For example, light could be directly coupled to a catheter 100a depicted in FIG. 1 using a customized hub.

In one embodiment, the increase or decrease in the temperature by the energetically activated nanostructure 30a, 30b, 30c, 30d comprises a change in temperature ranging from +/−50° C. to +/−1000° C. In another embodiment, the increase or decrease in the temperature by the energetically activated nanostructure 30a, 30b, 30c, 30d comprises a change in temperature ranging from +/−250° C. to +/−500° C. The increase in temperature may be from a base temperature, i.e., the temperature before activation of the energetically activated nanostructure 30a, 30b, 30c, 30d, that ranges from 20° C. to 40° C. The increase or decrease in the temperature by the energetically activated nanostructure 30a, 30b, 30c, 30d comprises a time period ranging from 0.1 seconds to 5.0 seconds per cycle. In one embodiment, the frequency in the increase or decrease in the temperature by the energetically activated nanostructure 30a, 30b, 30c, 30d ranges from 0.2 hertz to 100 hertz.

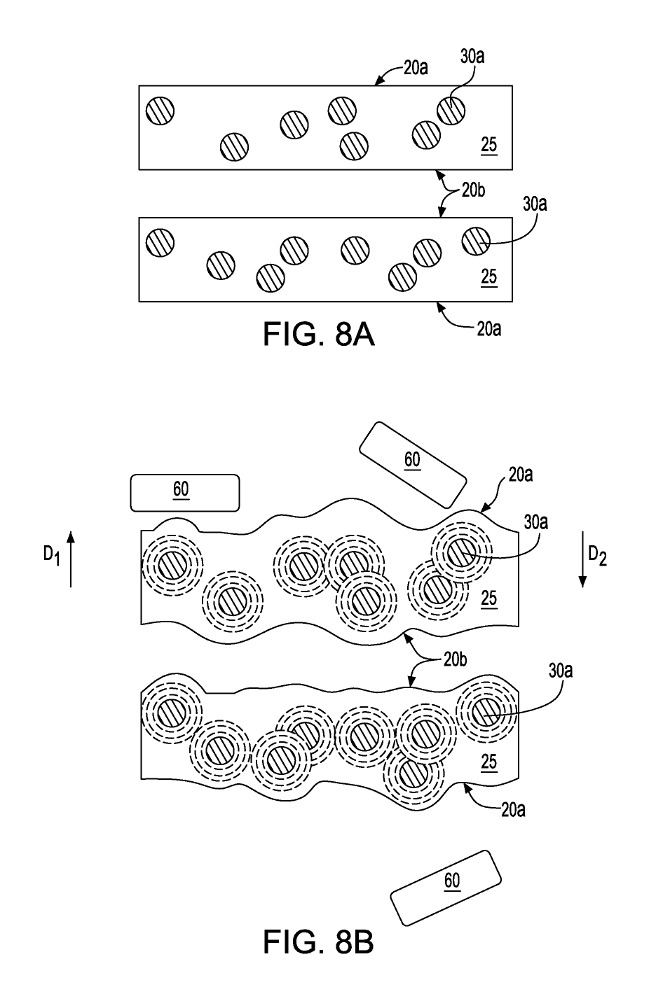

The increase or decrease in the temperature of the energetically activated nanostructure 30a, 30b, 30c, 30d, modifies at least one of a chemical and mechanical property of the base material 25, 35 to obstruct fouling. FIGS. 8A and 8B illustrate one embodiment of the mechanical motion used to obstruct fouling of a composite structure 15 as depicted in FIG. 2. FIGS. 8A and 8B are representative of the cross-sectional view of the lumen of a catheter. FIG. 8A depicts the geometry of the lumen before activation of the energetically activated nanostructure 30a. FIG. 8B depicts the geometry of the lumen during and/or following the activation of the energetically activated nanostructure 30a. In one embodiment, the increase and/or decrease in temperature of the nanoparticles of the energetically activated nanostructure 30a cause an expansion or contraction of the base material 25. In some embodiments, the expansion of the base material 25 may be as great as a 5% increase (+) in dimension D1. In further embodiments, the expansion of the base material 25 may be as great as a 1% increase (+) in dimension D1, as measured from original position of the surface 20a, 20b before the activation of the energetically activated nanostructure 30a. The surface 20a, 20b that experiences the expansion or contraction, i.e., mechanical motion, may be the interior surface 20b and/or exterior surface 20a of the lumen. In some embodiments, the contraction of the base material 25 in response to cooling after temperature increases induced by the energetically activated nanostructure 30a may be as great as a 5% decrease (−) in dimension D2, as measured from original position of the surface 20a, 20b before the activation of the energetically activated nanostructure 30a. In further embodiments, the contraction of the base material 25 in response to cooling after temperature increases induced by the energetically activated nanostructure 30a may be as great as a 1% decrease (−) in dimension D2, as measured from original position of the surface 20a, 20b before the activation of the energetically activated nanostructure 30a.

As the application of the energetically activated nanostructure 30a is cycled, the corresponding expansions and contractions are also cycled. The mechanical motion D1, D2 of the surface 20a, 20b prevents microorganisms 60, such as pathogens, from colonizing the structure. In another embodiment, the mechanical motion D1, D2 of the surface 20a, 20b may also destroy biofilms that are present on the structure. Examples of microorganisms 60 and biofilms that may be treated using the compositions, structures and method of the present disclosure include Staphylococcus aureus, Staphylococcus epidermidis, Pseudomonas, Streptococcus firidans, Escherichia coli and other bacteria or fungi or a combination thereof.

Although the mechanical motion that obstructs fouling is described with reference to the composite material 15 that is depicted in FIG. 2, the energetically activated nanostructures 30b, 30c, 30d that are depicted in FIGS. 3, 4A and 4B may also produce a mechanical motion that obstructs fouling when activated using an optical (light) wave source. For example, the base material 25 of the sheets in the laminated structure may experience a change in dimension, e.g., expansion and/or contraction, in response to changes provided by the energetically activated nanostructure 30b when subjected to optical waves. Further, the nanoparticles of the energetically activated nanostructure 30c in the coating 55 can cause mechanical motions in the matrix 26 of the coating 55 or the base structure 35 that the coating 55 is present on, as depicted in FIG. 7A. In yet another embodiment, the monolayer of nanoparticles that provides the energetically activated nanostructure 30d may cause a change in dimension, e.g., expansion and/or contraction, to the base structure 35 on which the energetically activated nanostructure 30d is formed. In one example, in which metal nanoparticles are embedded in a plastic catheter, nanoparticle heating and cooling causes expansions and contractions of the plastic component of the catheter that translates to mechanical surface motions. Such local changes in surface properties are disruptive to the adsorption of fouling agents. In each of the above-described embodiments and examples, the energetically activated nanostructure 30a, 30b, 30c, 30d may be directly, or remotely, activated.

In another embodiment, the chemical properties of the base material 25 containing the energetically activated nanostructure 30a, 30b of the structures that are depicted in FIGS. 2 and 3 may be modified to obstruct fouling in response to a stimuli of optical waves. For example, in response to the temperature changes resulting from the application of optical waves to the energetically activated nanostructure 30a, 30b, the base material 25 may experience a phase change, chemical reaction, or combination thereof that obstructs fouling. For example, when the temperature of the energetically activated nanostructure 30a, 30b, 30c, 30d increases, e.g., increases to 1000° C., the surrounding base material 25 may experience localized softening and/or melting. For example, some polymers that are suitable for the base material 25 have a melting temperature of 300° C., and have a softening temperature of 80° C. As the temperature of the base material 25 that is adjacent to the energetically activated nanostructure 30a, 30b, 30c, 30d increases in temperature beyond the softening temperature, and the melting temperature, chemical remodeling may occur which can obstruct fouling of the base material 25. The chemical remodeling can change the surface charge of the base material 25, which can obstruct fouling of the base material 25.

Similarly, the structures depicted in FIGS. 4A and 4B may also experience a change in chemical properties in response to the activation of the energetically activated nanostructure 30c, 30d that is present therein. Referring to FIG. 7A, either the matrix 26 of the coating 55, or the base structure 35 that the coating 55 is present on, may experience a change in chemical properties that results from an increase or decrease in the temperature of the energetically activated nanostructure 30c when subjected to an optical stimuli. Referring to FIG. 7B, the base structure 35 underlying the monolayer of the energetically activated nanostructure 30d may experience a chemical response, such as a phase change or chemical reaction, that obstructs fouling when the energetically activated nanostructure 30d is subjected to an optical stimuli.

In another embodiment, the stimuli that effectuates the increase or decrease in the temperature of the energetically activated nanostructure 30a, 30b, 30c, 30d that is depicted in FIGS. 2-5 is a magnetic field generated by an alternating current. In this embodiment, the nanoparticles that provide the energetically activated nanostructure 30a, 30b, 30c, 30d may be superparamagnetic.

In superparamagnetic materials, the nanoparticles consist of a single magnetic domain, wherein heating can be attributed to friction of the particle rotating in the magnetic field or to Néel relaxation where energy applied to the nanoparticle, by alternating magnetic field, allows the magnetic moment in the particle to overcome the energy barrier. This energy is then dissipated as heat when the particle moment relaxes to its equilibrium orientation. One example, of a nanoparticle composition that experiences an increase or decrease in temperature in response to a magnetic field is iron oxide (Fe₂O₃). The magnetic filed may be formed using induction coils. The strength of the magnetic field used to heat the nanoparticles of the energetically activated nanostructure 30a, 30b, 30c, 30d may range from 0.05 kiloampere/meter (kA/m) to 15 kiloampere/meter (kA/m). In one embodiment, the frequency of the magnetic field ranges from 0.05 MHz to 1.5 MHz. The increase or decrease in the temperature of the energetically activated nanostructure 30a, 30b, 30c, 30d that results from the application of a magnetic filed may also modify at least one of a chemical property of the base material 25, 35 to obstruct fouling.

While the present disclosure has been particularly shown and described with respect to preferred embodiments thereof, it will be understood by those skilled in the art that the foregoing and other changes in forms and details can be made without departing from the spirit and scope of the present disclosure. It is therefore intended that the present disclosure not be limited to the exact forms and details described and illustrated, but fall within the scope of the appended claims.

What is claimed:

1. A composite comprising:
a matrix phase of a first material composition; and
a dispersed phase of an energetically activatable nanostructure of a second material composition intermixed with the matrix of the first material composition, the dispersed phase of the energetically activated nanostructure when activated modifies the matrix phase to obstruct fouling of the composite.

2. The composite of claim 1, wherein the first material composition is a polymer.

3. The composite of claim 1, wherein the matrix phase is a catheter, shunt, artificial joint, dental implant, cosmetic implant and combination thereof.

4. The composite of claim 1, wherein the dispersed phase of the energetically activated nanostructure comprise nanoparticles having a longest axis of 1000 nm or less, and are comprised of a metal selected from the group consisting of gold, silver, copper, iron, palladium, platinum and a combination thereof.

5. The composite of claim 1, wherein the energetically activated nanostructure comprises nanoparticles having a geometry selected from the group consisting of spherical, platelet, rod-shaped and a combination thereof.

6. The composite of claim 4, wherein the concentration of the nanoparticles in the dispersed phase of the energetically activated nanostructure ranges from 1×10⁹ nanoparticles/cm³ to 1×10¹⁵ nanoparticles/cm³.

7. The composite of claim 1, wherein the composite is a laminate and the dispersed phase of the energetically activated nanostructure is present in a sheet geometry.

8. The composite of claim 1, wherein the dispersed phase of the energetically activated nanostructure is activated optically or by an alternating magnetic field.

9. The composite of claim 8, wherein modification of the matrix phase comprises a chemical change or a physical change of the first material composition.

10. The composite of claim 9, wherein the physical change of the first material composition comprises mechanical motion.

11. The composite of claim 9, wherein the chemical change comprises a phase change or chemical reaction.

12. The composite of claim 8, wherein the dispersed phase of the energetically activated nanostructure is activated optically, wherein in response to optic waves the energetically activated nanostructure causes an expansion or contraction of the first material composition of the matrix phase that modifies the matrix phase to obstruct fouling of the composite.

13. A coated structure comprising:
a geometry of a base material composition; and
a coating of an energetically activated nanostructure on a surface of the geometry of the base material composition, wherein when activated the energetically activated nanostructure modifies the geometry of the base material composition to obstruct fouling of the coated structure.

14. The coated structure of claim 13, wherein the base material composition is a polymer.

15. The coated structure of claim 13, wherein the geometry of the base material composition is a catheter, shunt, artificial joint, dental implant, cosmetic implant and combination thereof.

16. The coated structure of claim 13, wherein the coating of the energetically activated nanostructure comprises nanoparticles with a longest axis of 1000 nm or less.

17. The coated structure of claim 16, wherein the nanoparticles are comprised of a metal selected from the group consisting of gold, silver, copper, iron, palladium, platinum and a combination thereof.

18. The coated structure of claim 17, wherein said coating of the energetically activated nanostructure comprises a monolayer film of nanoparticles.

19. The coated structure of claim 17, wherein the coating of the energetically activated nanostructure is activated optically or by magnetic field.

20. The coated structure of claim 18, wherein the geometry of the base material composition when modified comprises a chemical change or a physical change of the first material composition.

21. A method to treat fouling comprising:
providing a structure composed of a first component of a base material and a second component of an energetically activated nanostructure; and
applying a stimuli to the structure that energetically activates the nanostructure and modifies at least one of a chemical and physical property of the base material to obstruct fouling.

22. The method of claim 21, wherein the structure is selected from the group consisting of a catheter, shunt, artificial joints, dental implant, cosmetic implant and combination thereof.

23. The method of claim 21, wherein the energetically activated nanostructure comprises nanoparticles each having a longest axis of 1000 nm or less.

24. The method of claim 23, wherein said nanoparticles have a composition that comprises iron, gold, cobalt, silver, copper, palladium, platinum, lanthium, strontium, manganese or oxides and combinations thereof.

25. The method of claim 21, wherein the providing of the structure comprises:
forming a nanoparticle suspension;
mixing the nanoparticle suspension with a carrier material to form a coating composition; and
depositing a coating of the coating composition on the first component of the base material, wherein the coating provides the second component of the energetically activated nanostructure.

26. The method of claim 21, wherein the providing of the structure comprises:
forming a nanoparticle suspension;
mixing the nanoparticle suspension with a polymer melt; and
forming the polymer melt including nanoparticles from said nanoparticle suspension into said structure, wherein the polymer melt provides the first component of the base material, and the nanoparticles from said colloidal nanoparticle suspension provide the second component of the energetically activated nanostructure.

27. The method of claim 21, wherein the second component of an energetically activated nanostructure is a monolayer of functionalized nanoparticles bonded to a reactive surface of the first component of the base material.

28. The method of claim 21, wherein the stimuli that effectuates the increase or decrease in a temperature of the energetically activated nanostructure comprises applying a magnetic field generated by an alternating current.

29. The method of claim 28, wherein a frequency of the magnetic field ranges from 0.05 MHz to 1.5 MHz.

30. The method of claim 29, wherein strength of the magnetic field ranges from 0.05 kiloampere/meter (kA/m) to 15 kiloampere/meter (kA/m).

31. The method of claim 21, wherein the stimuli that effectuates the increase or decrease in a temperature of the energetically activated nanostructure comprises applying near infrared (NIR) optical waves to the energetically activated nanostructure.

32. The method of claim 21, wherein the stimuli that effectuates the increase or decrease in a temperature of the energetically activated nanostructure comprises applying optical waves to the energetically activated nanostructure having a wavelength ranging from 300 nm to 700 nm.

33. The method of claim 21, wherein the increase or decrease in the temperature comprises a change in temperature ranging from +/−50° C. to +/−1000° C.

34. The method of claim 21, wherein the increase or decrease in the temperature comprises frequency ranging from 0.2 hertz to 100 hertz.

35. The method of claim 21, wherein the mechanical properties of the base material that are modified to obstruct fouling comprise mechanical motions including expansion and contraction of the base material up to +/−5%.

36. The method of claim 21, wherein said obstruct fouling comprises preventing microorganisms from colonizing the structure, destroying biofilms present on the structure, and a combination thereof.
